(19) United States
(12) Patent Application Publication
(10) Pub. No.: US 2019/0336457 A1
(43) Pub. Date: Nov. 7, 2019

(54) COMPOSITIONS AND METHODS FOR ALLEVIATING PAIN

(71) Applicant: LEADING EDGE PHARMS INC., Henderson, NV (US)

(72) Inventors: David J. Chadwick, Carlsbad, CA (US); Daniel D. Holsworth, SAN DIEGO, CA (US)

(21) Appl. No.: 16/388,507

(22) Filed: Apr. 18, 2019

Related U.S. Application Data
(60) Provisional application No. 62/659,567, filed on Apr. 18, 2018.

(51) Int. Cl. A61K 31/05; A61K 36/886; A61K 9/127; A61K 9/00; A61K 47/24

(52) U.S. Cl. CPC A61K 31/05; A61K 36/886; A61K 47/24; A61K 9/0014; A61K 9/127

(57) ABSTRACT

A topical composition including cannabidiol (CBD) and Aloe Vera in the form of nanoencapsulations with an average size of between about 50 nm to about 500 nm. Methods of using the topical composition for treating skin conditions of a subject are also disclosed.

### CROSS REFERENCE TO RELATED APPLICATION

This application claims the benefit of priority to U.S. Provisional Application No. 62/659,567, filed Apr. 18, 2018, the disclosure of which is incorporated herein by reference in its entirety.

### BACKGROUND

Opioids have been routinely prescribed for treating many types of chronic and acute pain. However, opioids have well-known pharmacodynamic profiles associated with a significant number of side effects and complications. With the increasing prescription of opioid drugs, millions of people are affected by the side effects of opioids and death rates from overdoses have quadrupled since 1999, numbering in the tens of thousands annually, according to the National Institute on Drug Abuse (NIDA).

A growing body of evidence suggest that cannabinoids—chemical components that can be found in Cannabis plants—can be effective in alleviating and controlling pain. Preclinical and clinical studies of Cannabis extracts have suggested that the use of these substances may be useful to treat diverse diseases, including those related with acute or chronic pain. Indeed, Sativex, a Cannabis sativa extract, was granted approval in Canada for the relief of neuropathic pain in multiple sclerosis.

The botanical genus, Cannabis, is known to produce more than 480 different chemical compounds, among which approximately 85 distinct entities are known as cannabinoids. Cannabinoids are chemical analogs that have structural similarity to Tetrahydrocannabinol (THC) and Cannabidiol (CBD), the two main active ingredients in Cannabis. While THC has a range of medical benefits, it is psychoactive and can cause impaired motor skills and other long term side effects. CBD, on the other hand, is a non-psychoactive component of Cannabis. CBD has been shown to relieve pain, reduce inflammation, and possess anti-convulsive properties.

The cutaneous endocannabinoid system (ECS), includes the endocannabinoids—enzymes involved in the biosynthesis and metabolism of the endocannabinoids and their two G-protein—coupled receptors, CB1 and CB2. Endocannabinoids are bioactive lipid mediators, produced in virtually all cell types and organs, which exert biological activity similar to cannabis. N-Arachidonoylethanolamine (AEA) and 2-Arachidonoylglycerol (2-AG) are the two most-studied endocannabionoids. They exert their biological effects similar to cannabis via activation of CB1 and CB2. CBD is shown to enhance N-arachidonoylethanolamine (AEA) signaling by preventing AEA degradation within the cutaneous endocannabinoid system. Prolonged signaling of AEA has been shown to decrease inflammation, pain, and the itch sensation. Furthermore, CBD can also act as an agonist of the Transient Receptor Potential Vanilliod subfamily V member 1 (TRPV1) nonselective cation channel. The function of the TRPV1 channel is to provide analgesic or pain relief.

CBD is highly lipophilic with poor pharmacokinetic properties, rendering it a poor candidate for oral or intravenous administration. It is not absorbed well when administered systemically (i.e., oral dosage), and its aqueous solubility is too low for intravenous administration.

### SUMMARY

It is thus an object of the invention to provide topically administered CBD-based products with good permeability for alleviating pain and other symptoms of a diseased or traumatic and/or acute skin condition.

In one aspect, a topical composition is provided, which comprises cannabidiol (CBD), wherein the CBD is present in the form of nanoencapsulations, and wherein the size of the nanoencapsulations is between about 50 nm to about 500 nm. The composition can further include Aloe Vera.

In some embodiments, the average size of the nanoencapsulations is between about 50 nm to about 200 nm.

In some embodiments, the average size of the nanoencapsulations is between about 80 nm to about 120 nm.

In some embodiments, the composition further includes ethanol. In some embodiments, the composition comprises at least one phospholipid.

In some embodiments, the CBD is present in the amount of from about 0.01% to about 5% by weight of the total composition. In some embodiments, the amount of CBD is from about 0.5% to about 4% by weight of the total composition. In other embodiments, the amount of CBD is from about 1.0% to about 2% by weight of the total composition.

In some embodiments, the Aloe Vera is present in the amount of between about 0.01% and about 10% of the total composition. In some embodiments, the amount of Aloe Vera is from about 0.05% to about 5% by weight of the total composition. In some embodiments, the amount of Aloe Vera is from about 0.1% to about 4% by weight of the total composition.

In some embodiments, the composition is essentially free of tetrahydrocannabinol (THC).

In another aspect, a method of alleviating pain of a subject is provided, the method comprising administering a therapeutically effective amount of a topical composition described herein. The pain may be associated with an inflammatory skin condition or a traumatic injury of the skin.

In another aspect, a method of treating a symptom of a diseased skin condition of a subject is provided, which comprises administering to the subject a therapeutically effective amount of a topical composition described herein. The diseased skin condition may be an inflammatory skin condition.

### BRIEF DESCRIPTION OF THE DRAWINGS

FIGS. 1A and 1B show particle size distribution of a CBD composition before micro-fluidization and after micro-fluidization, respectively.

FIG. 2 is a plot showing the rate of flux of CBD through the skin in a test using a composition of the present invention and a control agent.

FIG. 3 is a plot showing the amount of CBD delivered in 4, 10, and 24 hr periods via transdermal application in a test using a composition of the present invention and a control agent.

FIG. 4 is a plot showing the amount of CBD found remaining in the epidermis and dermis after 24 hrs in the Franz diffusion cell in a test using a composition of the present invention and a control agent.

### DETAILED DESCRIPTION

In one aspect, the present application provides a topical composition comprising CBD. The compositions can further comprise of Aloe Vera, ethanol, surfactants such as phospholipids (such as lecithin), and other excipients. The remainder and bulk of the composition can be water. The CBD is present in the form of nanoencapsulations (micelles or liposomes) in the water-based suspension, and the average size (or diameter) of the nanoencapsulations can be between about 50 nm to about 500 nm. For example, the average size of the nanoencapsulations can be between about 50 nm to about 200 nm.

As used herein, the term “about” or “approximately” means within an acceptable error range for the particular value as determined by one of ordinary skill in the art, which will depend in part on how the value is measured or determined, i.e., the limitations of the measurement system. For example, “about” can mean a range of up to 15%, preferably up to 10%, more preferably up to 5% of a given value.

CBD can be carried in the hydrophobic bilayers of the nanoencapsulation formed by the phospholipids. CBD exhibits multiple mechanisms of action within the skin. CBD enhances N-arachidonoylethanolamine (AEA) signaling by preventing AEA degradation within the cutaneous endocannabinoid system. Prolonged signaling of AEA has been shown to decrease inflammation, pain, and the itch sensation. CBD also acts as an agonist of the Transient Receptor Potential Vanilliod subfamily V member 1 (TRPV1) nonselective cation channel. The function of the TRPV1 channel is to act as an analgesic (i.e., a pain reliever).

CBD has been commercially available from various vendors. While many natural extracts of Cannabis contain both CBD and THC, only CBD is used for this invention, which avoids the side psychoactive effects of THC. Thus, in some embodiments, the composition is essentially free of THC. By “essentially free”, it is meant that THC is in an amount that is less than 1 millionth by weight of the total composition. CBD can be isolated via an extraction process that utilizes liquid carbon dioxide as the solvent. This process allows isolation of only the hydrophobic components of the plant, primarily CBD oil.

Aloe Vera as used herein is a natural plant extract derived from the leaves of one or more species of Aloe. The aloe plant can be squeezed, dried and crushed to produce powder form extract. In some embodiments, the Aloe Vera (dry powder form) used is Emerald Aloe Vera extract, which has been shown to be an effective cyclooxgenase-2 (COX-2) inhibitor, useful in reducing pain and inflammation. In addition, it contains anthraquinones that have anti-oxidant and anti-inflammatory activity.

CBD can reduce inflammation, pain and itch by enhancing N-arachidonoyletholamine (AEA) signaling in the epidermis and epidermal/dermal junction of the skin. Further, CBD acts as an agonist of the TRPV1 non-selective cation channel, that is responsible for relieving pain signaling. Aloe Vera contains many vitamins, enzymes, sugars, lignin, minerals, saponins, salicyclic acid and amino acids. These chemicals function as anti-oxidants, anti-inflammatory agents and analgesics. These chemicals also function through different mechanisms of action such as Cox-2 inhibition. Bradykinase and C-glucosyl chromone in Aloe Vera have shown to be effective anti-inflammatory agents when dosed alone. Aloe Vera components can be present in the shells or the interior of the nanoencapsulations in the formulation.

Thus, when Aloe Vera is administered with CBD, a variety of mechanisms of action are in play when relieving pain and inflammation. Therefore, CBD administered with Aloe Vera, via the form of nanoencapsulation, can give a synergistic, multiple component approach to alleviating pain and inflammation in the skin.

The outermost layer of the epidermis, the stratum corneum (SC), consists of denucleated, nonliving, flattened cells called corneocytes. There are ten to 25 layers of stacked corneocytes, which are nonhydrated cells lying parallel to the skin surface. The SC layers are united by SC lipid bilayers assembled into a “brick and mortar” arrangement. Below the SC, the remainder of the epidermis is viable epidermis, which is a region for drug binding, metabolism, active transport, and surveillance. The viable epidermis is separated from the dermis at the dermal-epidermal junction. The SC acts as the primary skin barrier. The “brick and mortar” arrangement of cells of the SC allows small hydrophobic structures to penetrate the stratum corneum and get to at the least the top of the epidermis. The hydrophilic nature of the micelles/liposomes aids in carrying the CBD/Aloe slightly deeper since the epidermis hydrophilicity increases as the dermis and basement membrane is approached.

In some embodiments, the average size of the CBD nanoencapsulations can be between about 80 nm to about 120 nm. This size allows the liposomes (or micelles) of CBD to traverse the epidermal layer and yet not the dermal layer to any appreciable extent. This “epidermal/dermal” junction area, is a desired location (where the nerve endings reside) and where the liposomes are concentrated when carrying CBD and Aloe Vera. Particles larger than 120 can have difficulty getting past the stratum corneum and particles that are smaller than 80 nm can have a tendency to travel to the dermis and risk systemic exposure of CBD.

The compositions of the present invention can be made into a cream, a gel, oil, spray, serum, or as a part of a medical device suitable to apply on the skin of a subject (e.g., a human or another mammal) to treat or alleviate pain, itchiness, or other symptoms associated with an inflammatory skin condition (e.g., dermatitis (eczema), rosacea, seborrheic dermatitis, and psoriasis) or a traumatic or acute skin injury (e.g., abrasion). The compositions can be formulated to further include other common pharmaceutically acceptable carriers or excipients for topical applications.

The compositions of the present disclosure have been shown to mediate pain perception and inflammation, and indicate potential in the topical symptomatic treatment of the following conditions: neuropathic pain, burning feet syndrome, diabetic foot neuropathy, chronic low back pain, fibromyalgia syndrome, neck pain, post herpetic neuralgia, pain and inflammation of arthritis (hands, knees, joints); rheumatoid arthritis and osteoarthritis, trigeminal neuralgia (TN), pudendal neuropathy and pudendal nerve entrapment (PNE), sciatica pain, muscle strains, tendon injuries, plantar fasciitis, herniated disks; phantom limb pain following amputation, and dermatological disorders like psoriasis, eczema, dermatitis.

The compositions of the invention can be administered in a fashion, (i.e., amounts, concentrations, schedules), consistent with good medical practice. Factors for consideration in this context include the disorder being treated, the subject being treated, the clinical condition of the individual patient, the cause of the disorder, the site of delivery of the agent, the method of administration, the scheduling of administration, and other factors known to medical practitioners.

### Example 1. Preparation of a CBD Nanoencapsulation Composition (Liposomal CBD)

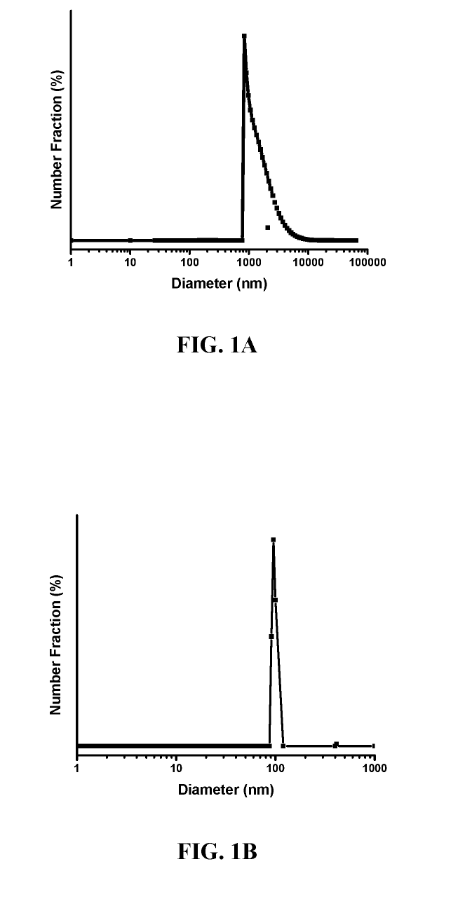

A CBD nanoencapsulation composition can be prepared as follows. 5-25 g CBD oil (which contains more than 99.98% CBD) is dissolved in ethanol (50-250 g), and phospholipids (20-150 g) (for example, phospholipon 85G (commercial product derived from Lecithin (plant)) are added at room temperature. Once dissolved, this is added to water (200-2000 mL) under vigorous homogenization conditions. Emerald Aloe Vera powder (5-100 g) and tetrasodium EDTA (0.5-5 g) is added directly to the mixture and homogenization continued for 15 minutes to 1 hour. The resulting emulsion is then subjected to a micro-fluidization process where the emulsion enters a micro-fluidization chamber and is forced into the interaction chamber at extremely high speed, driven by a high-pressure pump. As a result, the CBD oil is encapsulated into the phospholipids to form micelles or vesicles in the presence of Aloe Vera. The micro-fluidization process can effectively reduce the size, as well as the size distribution of the CBD droplets in the dispersions. In one example, and as shown in FIGS. 1A and 1B (Dynamic Light Scattering analysis of particle size), before micro-fluidization, the size of the CBD droplets was about 1798.3±405 nm, and after micro-fluidization, the size of the droplets became 96.5±3.2 nm. The reduction in size of the CBD droplets improves the permeability of the composition through skin, thereby boosting its effectiveness in pain relief and other beneficial effects.

### Example 2

In this example, a composition of the present invention was prepared, and its topical performance compared with CBD loaded in dimethylsulfoxide (DMSO) via dermal penetration testing (also known as percutaneous penetration). This test measures the absorption or penetration of a substances through the skin barrier and into the skin.

### Material:

Sample 1: Liposomal CBD (or CBD nanoencapsulation, test agent), prepared and processed by micro-fluidization according to the procedure outlined in Example 1. The average particle size of CBD nanoencapsulations is between 80 nm and 120 nm. The carrier of the CBD comprises of 85% water and 15% ethanol. The effective concentration of CBD in total composition is 1.5 wt %.

Sample 2: CBD in DMSO (positive control), CBD dissolved in pure DMSO, at a concentration of 1.5 wt % (out of the total composition). DMSO is widely known as an effective carrier of chemicals through the skin to the dermis for systemic exposure. The FDA allows a carrier to be comprised of 45% or less DMSO due to its toxicity.

### Procedure:

5 μL of each of Sample 1 and Sample 2 was added to the top of human cadaver skin (surface area: 0.55 cm²) using a Franz static diffusion cells (3 mL volume). The receptor volume is 3.3 mL and the receptor fluid consists of: phosphate buffered saline (PBS) with 4% hydroxypropyl-β-cyclodextrin and 1% of a surfactant consisting of polyoxyethylene vegetable-based fatty ether (derived from cetyl alcohol) and 0.01% sodium azide. Measurements of CBD permeation through the skin was taken at 3 time-points: 4 hr, 10 hr and 24 hr. After 24 hrs, CBD was also measured in the epidermis and dermis by tape stripping.

Each sample consisted of 1.5 wt % of CBD and was dosed at 5 uL. Each measurement was repeated 6 times (n=6).

### Results:

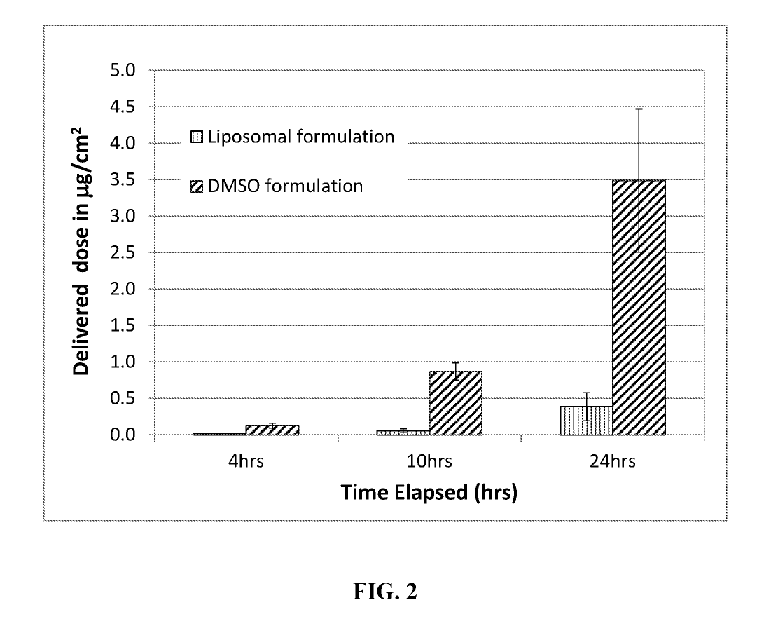

A dose of each solution (5 μL, n=6) was placed on the cadaver skin at room temperature and 0.3 mL of receptor solution taken at time points 4 hr, 10 hr, 24 hr. FIG. 2 shows the flux of CBD in each sample through the skin in units of μg/cm²/hr. For the liposomal formulation (CBD nanoencapsulation), CBD flux was marginal and most notable at the 24 hr period, with a flux rate of 0.03 μg/cm²/hr. For the CBD in DMSO, the rate of flux of CBD was about 0.2 μg/cm²/hr at the 24 hr time point. Thus, the rate of CBD flux through the skin in DMSO was about an order of magnitude higher than with the CBD nanoencapsulation. This rate relationship was similar for the 4 and 10 hr time-points.

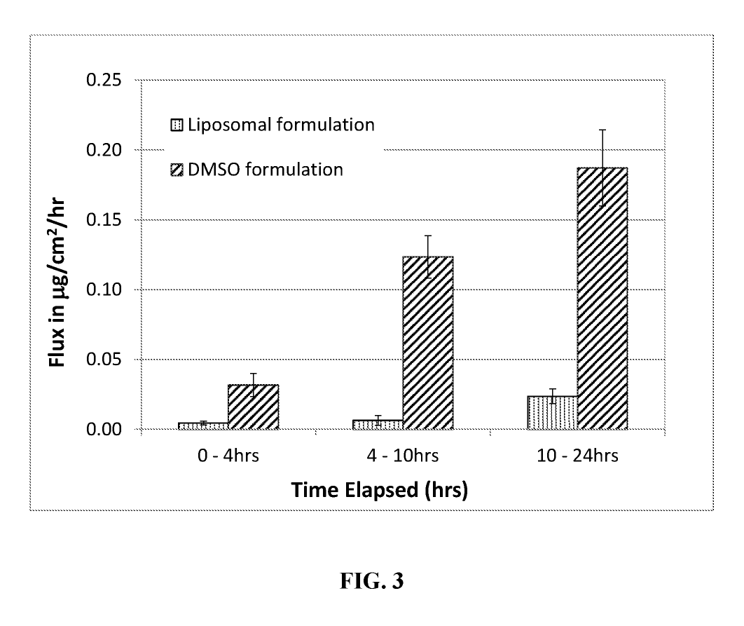

The amount of CBD in each sample delivered (m) per cm² area of skin is shown in FIG. 3. Once again, for “optimal” transdermal application of CBD, the CBD in DMSO solution transferred an average of 3.5 μg/cm² in a 24 hr period. For the liposomal CBD (CBD nanoencapsulation), about 0.4 μg/cm² was delivered in a 24 hr period. Thus, the CBD in DMSO form delivered a 9-fold higher amount of CBD than the CBD nanoencapsulation form. It is noted that the CBD in DMSO solution was used as a positive control and shows the optimal amount of CBD delivery of compound via transdermal mode for systemic exposure and to the epidermis and dermis.

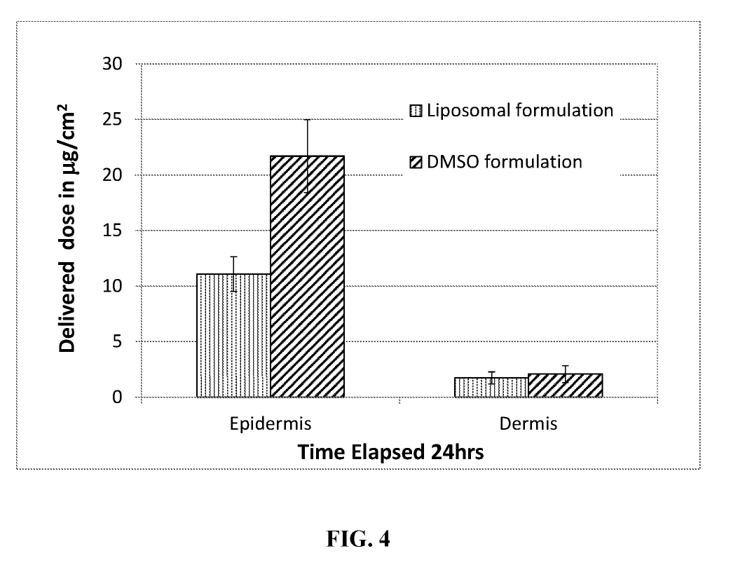

After 24 hrs, the skin was “tape stripped” to remove the epidermis from the dermis and then extracted to isolate amount of CBD found in each skin layer. FIG. 4 shows the amounts of CBD from each sample found in the epidermis and dermis. The majority of CBD from each solution was found in the epidermis and the epidermal-dermal junction.

These results show the CBD nanoencapsulation of the present invention can deliver sufficient compound-loading levels into a subject's skin with desired vesicle specific characters. The phospholipid enriched amphiphilic nature of the formulation can allow for the significant rate of CBD transfer across the stratum corneum and into the depths of the epidermis layer. Integration of phospholipid molecules with the skin lipids help to retain the CBD within the epidermis and epidermal/dermal layers of the skin thus, leading to the prolonged presence of compound molecules at the receptor site. Thus, the CBD nanoencapsulation form of the present invention has been demonstrated to be an effective form to deliver the CBD in the epidermis and epidermal/dermal junction where neuropathic nerve endings reside. The CBD is predominately deposited in the epidermis and epidermal/dermal junction and does not enter the systemic system to any extent (<1% after 24 hr exposure).

The findings of this Example show that the liposomal formulation of the present disclosure provides improved and localized CBD action in the skin, which is particularly useful for alleviating somatic nociceptive and peripheral neuropathic pain, discomfort and inflammation.

The present invention is not to be limited in scope by the specific embodiments described herein. It will be appreciated that the invention is susceptible to modification, variation and change without departing from the spirit thereof.

1. A topical composition comprising:
a) cannabidiol (CBD);
b) Aloe Vera;
wherein the CBD is present in the form of nanoencapsulations, and wherein the average size of the nanoencapsulations is between about 50 nm to about 500 nm.

2. The topical composition of claim 1, wherein the average size of the nanoencapsulation is between about 80 nm to about 120 nm.

3. The topical composition of claim 1, further comprising at least one phospholipid.

4. The topical composition of claim 1, wherein the CBD is present in the amount of about 0.01% to about 5% by weight of the total composition.

5. The topical composition of claim 1, wherein the Aloe Vera is present in the amount of between about 0.01% to about 10% of the total composition.

6. The topical composition of claim 1, wherein the composition is essentially free of tetrahydrocannabinol (THC).

7. A method of alleviating pain of a subject, comprising administering a therapeutically effective amount of a topical composition of claim 1 to the subject.

8. The method of claim 7, wherein the pain is associated with an inflammatory skin condition.

9. The method of claim 7, wherein the pain is associated with a traumatic injury of the skin.

10. A method of treating a symptom of a skin condition of a subject, comprising administering a therapeutically effective amount of a topical composition of claim 1 to the subject.

11. The method of claim 10, wherein the skin condition is an inflammatory skin condition.

12.-21. (canceled)
